(19) United States
(12) Patent Application Publication
(10) Pub. No.: US 2016/0374556 A1
(43) Pub. Date: Dec. 29, 2016

(54) TRANSCUTANEOUS READER FOR USE WITH IMPLANTABLE ANALYTE SENSORS

(71) Applicant: Profusa, Inc., South San Francisco, CA (US)

(72) Inventors: Arthur E. COLVIN, JR., Mt. Airy, MD (US); William A. MCMILLAN, La Honda, CA (US); Steven PULLINS, San Rafael, CA (US)

(21) Appl. No.: 15/193,711

(22) Filed: Jun. 27, 2016

Related U.S. Application Data
(60) Provisional application No. 62/184,785, filed on Jun. 25, 2015.
(60) Provisional application No. 62/239,536, filed on Oct. 9, 2015.

(51) Int. Cl. A61B 5/00; A61B 5/01; A61B 5/145

(52) U.S. Cl. CPC A61B 5/0017; A61B 5/0031; A61B 5/14503; A61B 5/01; A61B 5/14542; A61B 2562/166

(57) ABSTRACT

Some embodiments described herein relate to a reader having a distributed source of radiation and a photodetector. The photodetector can be operable to sense radiation (e.g., light) emitted by an implanted sensor. The distributed source of radiation can at least partially surrounds the photodetector. The distributed source of radiation generates a photon cloud of excitation radiation within the skin, which can substantially envelopes a sensor that is implanted within the skin at a depth that is on the order of a centimeter or less.

### CROSS-REFERENCE TO RELATED APPLICATION

This application claims the benefit of provisional U.S. Patent Application No. 62/184,785, filed Jun. 25, 2015, entitled “Sensor Interrogation System” and provisional U.S. Patent Application No. 62/239,536, filed Oct. 9, 2015, entitled “Transcutaneous Reader for Use with Implantable Analyte Sensors,” the contents of each of which are incorporated herein by reference in its entirety.

### BACKGROUND

Some embodiments described herein relate to implantable analyte sensors. More particularly, the some embodiments described herein relate to readers used in conjunction with such sensors to determine analyte levels transcutaneously. As used herein, a sensor is any device, chemical, or structure configured to emit a signal that can be correlated to a concentration or quantity of an analyte or parameter of interest.

Implantable sensors to determine the level of various substances within the human body are known in the art. For example, U.S. Pat. No. 6,304,766, which is incorporated herein by reference in its entirety, discloses a pill-shaped or bean-shaped sensing device that includes a light-emitter, a photodetector, and a transmitter that are embedded within a housing, with the housing being made from material such as an acrylic polymer that allows it to function as a waveguide. The housing is configured to be implanted under the skin, e.g., between the skin and subcutaneous tissue layers.

The housing of the device described in U.S. Pat. No. 6,304,766 has an external coating of material (“fluorescent indicator molecules”) that fluoresces when struck by radiation generated by the internal light-emitter, and fluorescent light emitted by the indicator molecules enters and is internally reflected within the housing. The photodetector is sensitive to the fluorescent light and generates a signal that corresponds to the amount of fluorescent light it detects. Furthermore, the extent to which the indicator molecules emit fluorescent light, and hence the extent to which the housing will be internally illuminated by the internally reflected fluorescent light, varies with concentration levels within the human body of a particular analyte of interest, e.g. glucose, oxygen, toxins, pharmaceuticals, etc. Therefore, the signal the photodetector generates will be indicative of the level of the analyte, at least within the tissues and/or bodily fluids in the vicinity of the device.

An external power supply and an external reader are used in conjunction with the sensing device disclosed in U.S. Pat. No. 6,304,766. The power supply powers the sensing device transcutaneously via an inductor, which induces a current within the sensing devices' circuitry to power the light-emitter, the photodetector, and the transmitter. The transmitter, in turn, transmits a signal that is indicative of the strength of the signal the photodetector generates and that is therefore indicative of the level of analyte being sensed. The transmitter signal can be, for example, further inductance, RFID-type signals, etc., and the reader is configured to sense the transmitted signal and enables the level of analyte to be determined.

Another implantable sensor is disclosed in U.S. Patent Publication 2012/0265034 to Wisniewski, the contents of which are incorporated by reference in its entirety. The sensors disclosed in this reference (referred to herein as “Wisniewski-type sensors”) do not include any internal electronics. Rather, the sensors disclosed in U.S. Patent Publication 2012/0265034 include various biocompatible scaffold structures, to foster the integration of tissue into the sensors, and various “sensing moieties” or indicator molecules that are supported by and distributed throughout the scaffold structures. The indicator molecules produce a detectable photonic signal in response to excitation radiation (e.g., light), and the amplitude (in some cases) or decay properties (in other cases) of the response signal when excitation radiation is applied and/or removed varies as a function of analyte concentration to which the sensor is exposed.

With sensors according to U.S. Patent Publication 2012/0265034, an external “reader” is positioned over the area where the sensor is implanted, and a discrete light-source on the reader emits light of a specific wavelength that penetrates through the skin to the sensor. Upon excitation by the specific wavelength of light, each excited indicator molecule will, in turn, radiate (emit) light of a longer, lower-energy wavelength, some of which will pass out of the skin. A photodetector within the reader, which is offset from the reader light-source as well as the implantable sensor, responds to the light passing out of the skin and generates a signal, the amplitude of which corresponds to the amount of light emanating from the implanted sensor and making its way out of the skin. By observing the amplitude of the light emitted by the indicator molecules, the concentration of the particular analyte of interest can be determined.

Importantly, each emission point (indicator element) can radiate light in all directions, i.e., as a point source of emission light. Therefore, upon excitation of the implant (or a portion thereof), the luminescence of the implant radiates in all directions. However, tissue through which the luminescence passes tends to scatter the luminescent light. Furthermore, only the emission light which can be captured by the detector and converted to current or voltage is productive in generating useful signal information; any portion of the overall emission light which radiates out and is not captured by the photodetector is “unproductive” and effectively wasted in terms of providing a strong, clear (i.e., accurate) indication as to the concentration of the analyte of interest. As a result, signal-to-noise ratio is sub-optimal.

### SUMMARY

Some embodiments described herein relate to a reader having a distributed source of radiation and a photodetector. The photodetector can be operable to sense radiation (e.g., light) emitted by an implanted sensor. The distributed source of radiation can at least partially surrounds the photodetector. The distributed source of radiation can generate a photon cloud of excitation radiation within the skin, which can substantially envelopes a sensor that is implanted within the skin at a depth that is on the order of a centimeter or less. As a result, substantially the entire sensor is utilized in indicating analyte concentration. For example, the photon cloud can excite the entire length of the sensor, and the duration of sensor excitation can be shortened which can prolonging the useful life of the sensor (e.g., by reducing the rate of photobleaching of individual indicator molecules), as well as improving signal-to-noise ratio of the light signal that is generated by the sensor. It also allows the reader to work well using lower power levels of emitted stimulating radiation, thereby extending the useful life of the reader's batteries or a battery-recharge interval.

In specific embodiments, the reader may have an optical filter disposed over the photodetector, e.g., to allow light from the sensor to reach the photodetector while excluding light from the distributed source of radiation. For example, some embodiments described herein can include one or more features of embodiments described in U.S. Patent Application Pub. No. 2014/0275869, the disclosure of which is hereby incorporated by reference in its entirety. The distributed source of radiation may be provided by way of a plurality of individual sources of light or emitters, e.g., LEDs, which can be substantially evenly spaced (in terms of angular position) relative to the photodetector. As described in further detail herein, inter-source spacing can be sufficient for the orbs of light created within the body by each source to merge or overlap, i.e., to form a sensor-enveloping photon cloud, as addressed below.) Outside of the body (e.g., in the absence of scattering) the orbs of light may not overlap and/or may not overlap completely. Suitably, the reader includes a temperature sensor to determine skin temperature, and it is preferably configured to transmit data wirelessly.

In some embodiments, the photodetector can be coupled to a central portion of a reader substrate, such as a housing, backing plate, printed circuit board, or so forth. Two or more emitters can be coupled to a peripheral portion of the reader substrate. The substrate, the photodetector, and the emitters can be collectively referred to as a “reader.” Each emitter can produce a signal (e.g., light) that radiates from the emitter in a predefined pattern. For example, the emitter can produce a sphere, lobe, or cone of light such that each emitter illuminates increasing cross-sectional areas as the distance from the emitter increases. The emitters' illumination patterns and spacing on the substrate can be selected, tuned, or configured to interact with the sensor when the sensor is disposed at a particular implantation depth. For example, in some embodiments, the emitters can be collectively configured such that, in the absence of tissue-induced scattering, the illumination fields from the emitters do not overlap at the implantation depth. Similarly stated, the far-field illumination pattern of the group of emitters at the implantation depth may form a toroidal shape having a central dark zone when the light is not scattered or reflected by tissue. When the reader is used in conjunction with an implanted sensor, the tissue can cause the light transmitted by the emitters to scatter to produce a photon cloud that can illuminate an entire length of the sensor.

In some embodiments described herein one or more optical isolation members can be coupled to the reader substrate. For example, a perimeter optical isolation member can be operable to reflect at least a portion of light emitted from one or emitters back towards a central region of the reader. For example, as described in further detail herein, the reader can be configured such that the photodetector is placed directly over the implanted sensor and the emitters, which can result in the emitters being spaced some lateral distance away. Thus, the emitters may transmit a portion of their light to a region of the tissue that does not contain the sensor, effectively wasting that portion of the light. A perimeter optical isolation member can be operable to reflect at least a portion of light that would otherwise be wasted towards the sensor. In other instances, a lens, waveguide, or any other suitable optical element can be operable to focus light from the emitters towards the sensor.

Some embodiments described herein relate to a method of interrogating an analyte sensor (also referred to as simply “a sensor”) that is implanted within tissue of an organism's body. The method can involve substantially enveloping the sensor with a photon cloud of excitation radiation within the organism's body sufficient to cause indicator molecules on the sensor to exhibit a photo-reaction. Similarly stated, an entire length and/or an entire surface area or a substantial portion thereof can be exposed to radiation emitted by a reader. For example, the reader can have two or more emitters. Each emitter can emit a light. Each emitter can be configured to emit light having a far-field illumination pattern that has a pre-defined cross sectional area at an implantation depth (e.g., the depth at which the implant is implanted within the tissue.) The cross-sectional area and/or diameter of the illumination pattern from each emitter, in the absence of scattering and/or reflection can be less than a surface area and/or length (or other characteristic dimension) of the sensor. In addition or alternatively the illumination pattern from each emitter, in the absence of scattering and/or reflection may not overlap the illumination pattern for any other emitter, at least in a central region. When the reader is applied to the skin, the light emitted from the emitters can be scattered by the tissue within which the sensor is implanted. In this way, light that would not otherwise overlap and/or would not otherwise have a sufficient surface area to illuminate the sensor, can diffuse into a photon cloud that illuminates the entirety of the sensor. In response to being illuminated by light originating from the emitters, the sensor can generate an emission signal, which can be analyte dependent. A photodetector of the reader, which can be centrally located between the emitters and placed over the implant (e.g., such that the emitters are spaced a lateral distance from the implantation site), can detect the emission signal. The emission signal can have an intensity associated with the entire surface area and/or length being illuminated by light scattered from the emitters. The reader and/or a computing device, such as a smart phone, can process the signal detected by the photodetector and determine the concentration of the analyte of interest.

### BRIEF DESCRIPTION OF THE DRAWINGS

FIG. 1 is a schematic diagram illustrating a reader being used with an implanted sensor, according to an embodiment;

FIG. 2 is a plan view of the radiation-emitting (e.g., light-emitting) and radiation-detecting portion of a transcutaneous reader, according to an embodiment, and

FIG. 3 is a section view thereof, taken along line 3-3 in FIG. 2, showing additional electrical components of the reader, too;

FIG. 4 is a schematic section view of the radiation-emitting (i.e., light-emitting) and radiation-detecting portion of the transcutaneous reader illustrated in FIGS. 2 and 3, illustrating how radiation emitted thereby is aimed at or concentrated toward an implanted analyte sensor;

FIG. 5 is a diagram illustrating the arrangement and interconnection of electrical components of the transcutaneous reader shown in FIGS. 1-4; and

FIG. 6 is a schematic diagram illustrating an advantageous manner in which a transcutaneous reader interacts with an implanted analyte sensor.

### DETAILED DESCRIPTION

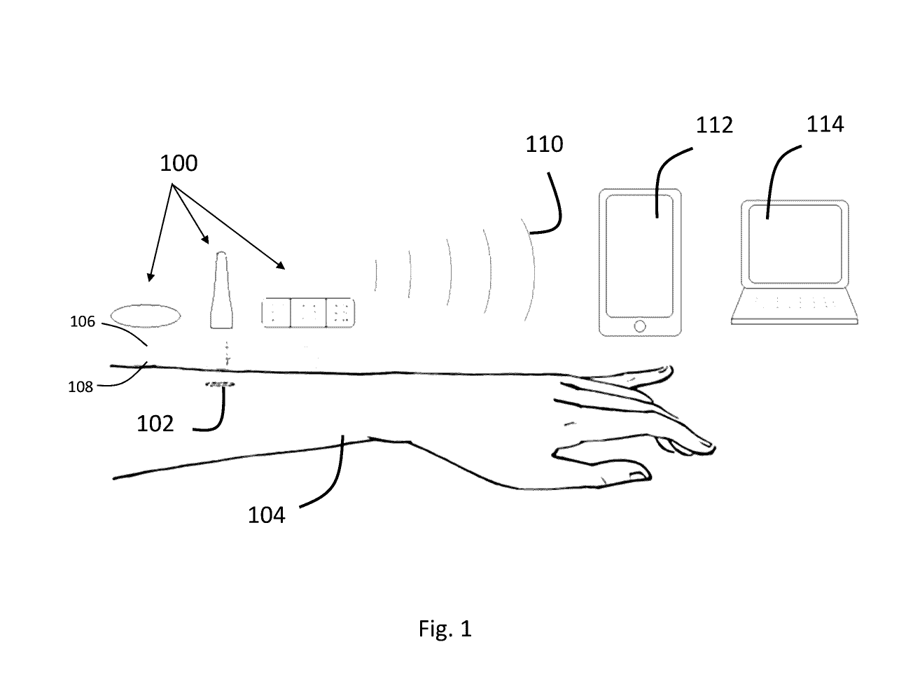

As illustrated in FIG. 1, a reader 100 can be used to query an analyte sensor 102 that is implanted within about a centimeter (e.g., a few millimeters, 2-5 mm, 1-10 mm, or any other suitable depth) beneath the surface of the skin on a user's forearm 104 (shown), abdomen, upper arm, leg, thigh, neck, foot, etc. depending on the particular analyte and/or tissue site of interest. As illustrated, the reader 100 could be configured as a patch, a wand, a bandage, or any of a number of other form factors, which can be brought into intimate contact with the surface of the user's skin. Regardless of its configuration, the reader 100 emits into the skin excitation radiation (e.g., light) 106 of a suitable wavelength to induce a photo- reaction by the indicator molecules of the sensor 102, and a portion of the light 108 emitted by the sensor 102 in response will exit from the skin. A photodetector within the reader 100 (not illustrated in FIG. 1) senses and responds to the emitted light 108 and sends a signal 110, such as a wireless signal, to a compute device such as a tablet computer or smartphone 112 running an app, or a desktop computer 114 running a full program. The compute device can be operable to query the reader 100, receive signals from the reader indicating a concentration of the analyte, and/or receive signals indicative of radiation detected by the photodetector. The compute device can calculate a concentration of an analyte based on a signal received by the photodetector and/or track the particular analyte. The amount, intensity, and/or decay properties of the emitted light 108 sensed by the photodetector corresponds to and can be used to determine the concentration of the analyte of interest, as explained in more detail below. Processing of the response signal can be performed onboard the reader 100 or by the external device 112 or 114.

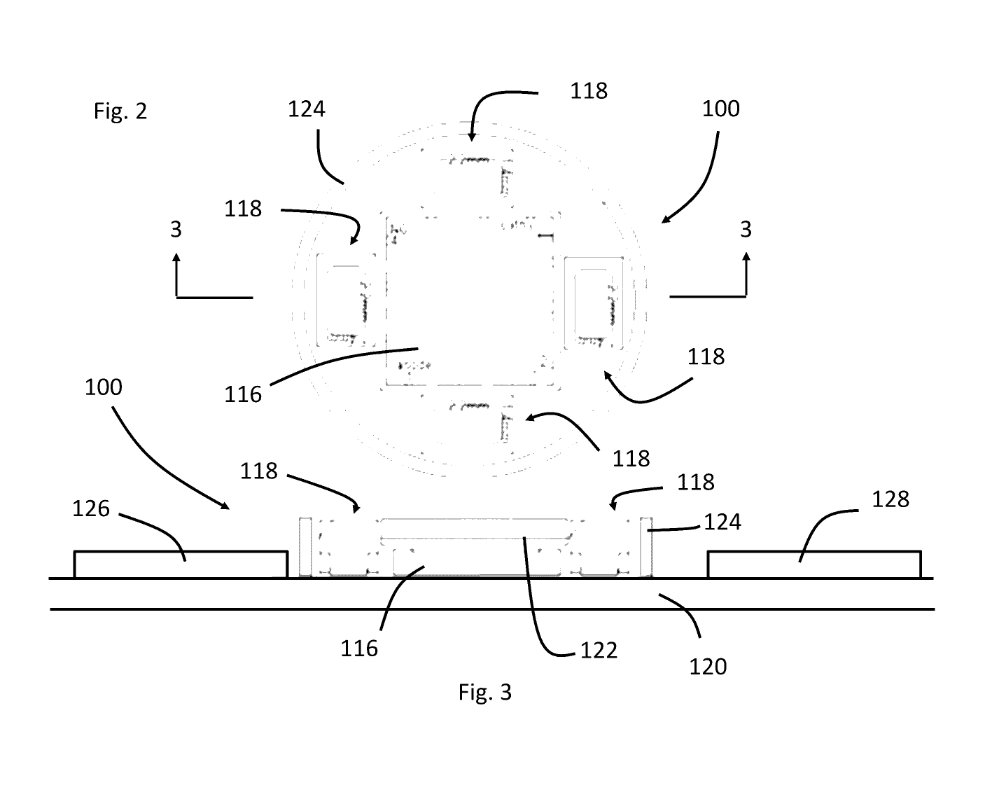

As illustrated in FIGS. 2-5, a reader 100 includes a photodetector 116 and a distributed source of excitation radiation (e.g., light) 118, which are can be mounted on a printed circuit board 120 or any other suitable substrate. The distributed source of excitation radiation 118 at least partially surrounds the photodetector 116. The photodetector 116 is essentially centered within the distributed source of excitation radiation 118. The distributed source of radiation 118 can include two or more LEDs, which can emit light at a wavelength that enables the light to penetrate through skin to the depth at which the sensor 102 is implanted. When the sensor 102 is illuminated, it can cause a photoreaction or excitation/emission condition by the indicator molecules on or within the sensor 102. For example, as best illustrated in FIG. 2, the distributed source of radiation 118 may be implemented with four “Super Red” LEDs, which are available from Vishay Intertechnology and emit light at 630 nm and which are evenly spaced from each other and distributed evenly around the photodetector 116, i.e., 90° apart from each other as centered on the photodetector 116. As for the photodetector 116, it suitably may be a silicon photomultiplier (SiPM) of the sort available from SensL, with an active area of 3 square millimeters. Any other suitable source of radiation and/or detector could also be used.

Suitably, the reader 100 further includes an optical band-pass filter 122 disposed over the active, light-receiving surface of the photodetector 116, which band-pass filter 122 allows light emitted by the sensor 102 to pass through to the photodetector 116 while screening out “stray” light emitted by the distributed source of radiation 118. The band-pass filter 122 may be constructed as a laminate using Schott RG715 colloidally colored glass (pass-through wavelength of approximately 715 nm and above) and Lee HT090 color filter film (pass-through wavelengths between approximately 450 nm and 575 nm) joined together with Epotek 301-2 optical epoxy.

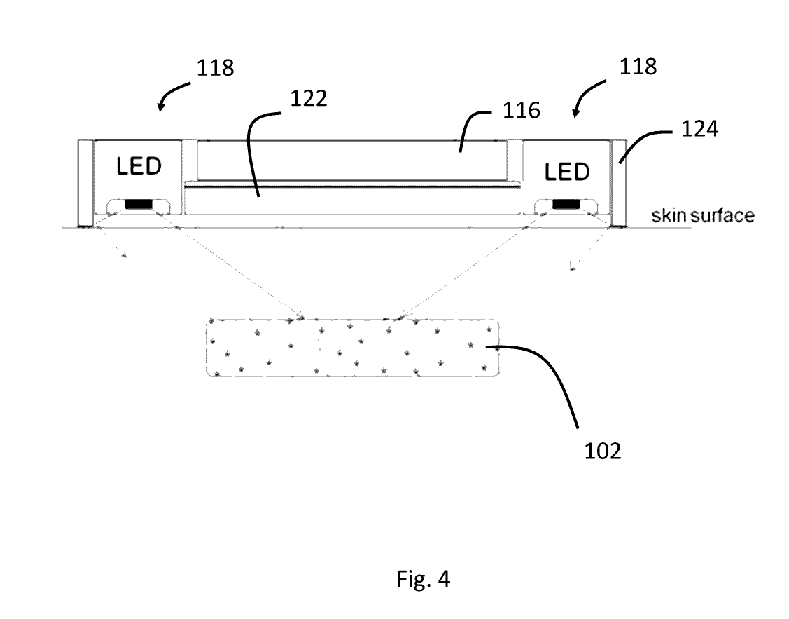

The photodetector 116, distributed sources of radiation 118, and optical band-pass filter 122 suitably are surrounded by a substantially reflective optical isolation ring 124, which serves to contain light emitted by the distributed source of radiation 118 and better concentrate it toward the sensor implant 102, as illustrated in FIG. 4. (FIG. 4 does not depict any scattering of light as it passes into/through the skin, which is addressed below.) The optical isolation ring 124 is suitably formed from dark gray or black PVC (or other material) and may have its inner surface smoothed or coated with reflective material to enhance the reflection/light-concentrating benefit of the isolation ring 124. After being mounted to the printed circuit board 120 and having the optical isolation ring 124 installed around them, the photodetector 116 and distributed source of radiation 118 may be partially encapsulated with black potting compound or, e.g., as available from MC Electronics, but with encapsulation leaving the light-emission points of the distributed source of radiation 118 and the light-receiving, active surface of the photodetector 116 exposed so as to emit and receive radiation (light), respectively. The potting compound (or other suitable material) can be operable to optically isolate the distributed source of radiation 118 from the photodetector 116, for example, preventing light from leaking from the distributed source of radiation 118 to the photodetector 116 without the light first passing through the tissue.

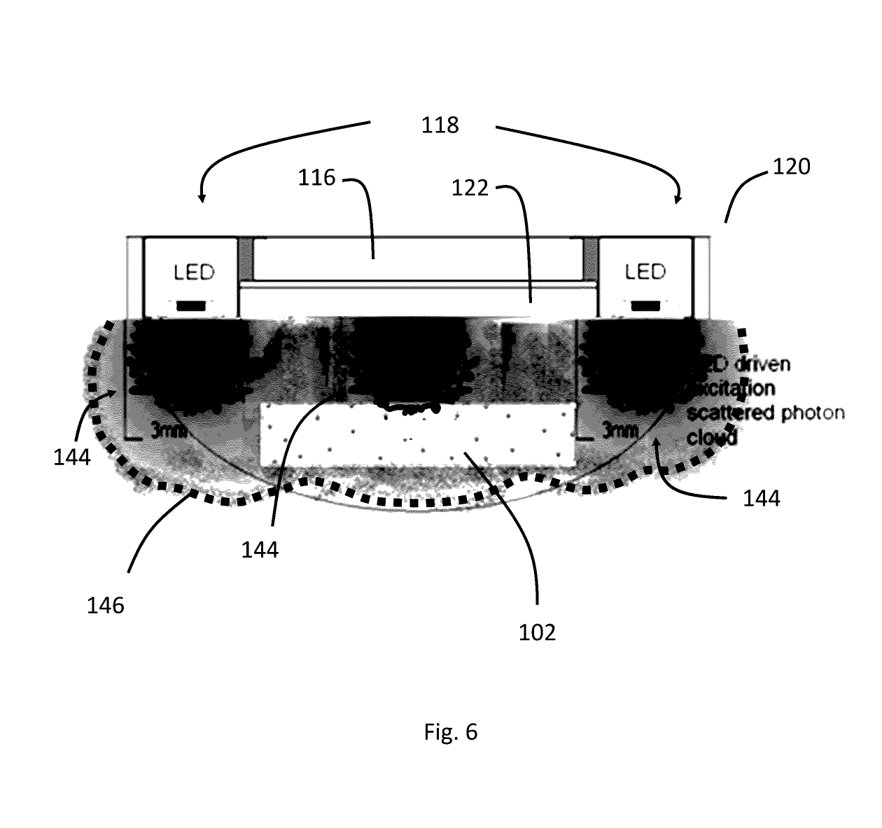

In some instances, each of the distributed source of radiation 118 can be configured to produce light with a particular far-field emission pattern. For example, each of the distributed sources of radiation 118 can produce an orb, lobe, or cone of light such that each distributed source of radiation 118 is configured to illuminate a particular cross-sectional area at a particular depth. In the absence of scattering and/or reflection, in some embodiments, each distributed source of radiation 118 may have a far-field emission pattern at a depth at which the implant is implanted (e.g., implantation depth, which can be approximately 2-5 mm) that is smaller than the implant. Similarly stated, in some embodiments, in the absence of scattering or reflection, no single emission source may have a cross sectional area and/or characteristic length at the implantation depth sufficient to illuminate an entirety of the implant. Moreover, as described herein, in some embodiments the distributed sources of radiation 118 may not be disposed directly over the sensor. Thus, in some embodiments, in the absence of scattering or reflection, the far-field emission pattern from each emission source need not be centered on the implant. In the presence of scatter, for example, induced by the tissue and/or reflectance e.g., from the optical isolation ring 124, the far-field emission patterns from the distributed sources of radiation 118 may merge into a photon cloud that substantially envelops the implant, for example as shown and described with reference to FIG. 6. The use of emitters that, in the absence of scattering and/or reflection produce far-field emission patterns at the implantation depth that are not large enough to illuminate an entirety of the sensor, that do not overlap each other and/or do not overlap at least in a central region, can reduce the power consumption of the reader and/or reduce photo-bleaching effects. Similarly stated, the distributed sources of radiation 118, in the absence of scattering, may produce a toroidal illumination pattern at the implantation depth with a darkened center (e.g., the center of the illumination patter, in the absence of scattering, may be at least 10%, 25%, 75% and/or any other suitable percentage darker than a peak brightness of the illumination pattern). Such embodiments can rely on scattering induced by tissue to fully illuminate the implant, which presents a contrast to known readers, which have typically viewed scattering as a impediment that has been compensated for by increasing illumination power.

The reader 100 suitably includes a temperature sensor 126, to measure skin temperature in the vicinity of the sensor 102. Skin temperature is used to correct calculations for quantum yield from the luminescent sensor 102. Suitably, the temperature sensor is provided as a TMP006, non-contact infrared digital temperature sensor available from Texas Instruments.

Operation of the various electronic components is controlled by a microprocessor 128, which is also mounted to the printed circuit board 120. Ideally, the microprocessor 128 includes built-in or integrated wireless capability to enable wireless transmission and receipt of data/operating commands between the reader 100 and the monitoring device, i.e., smartphone or tablet computer 112 or desktop computer 114. Thus, a Simblee™ RFD77101, Bluetooth Low Energy-enabled module with built-in ARM Cortex M0 microcontroller is the presently preferred device for use as the microprocessor 128.

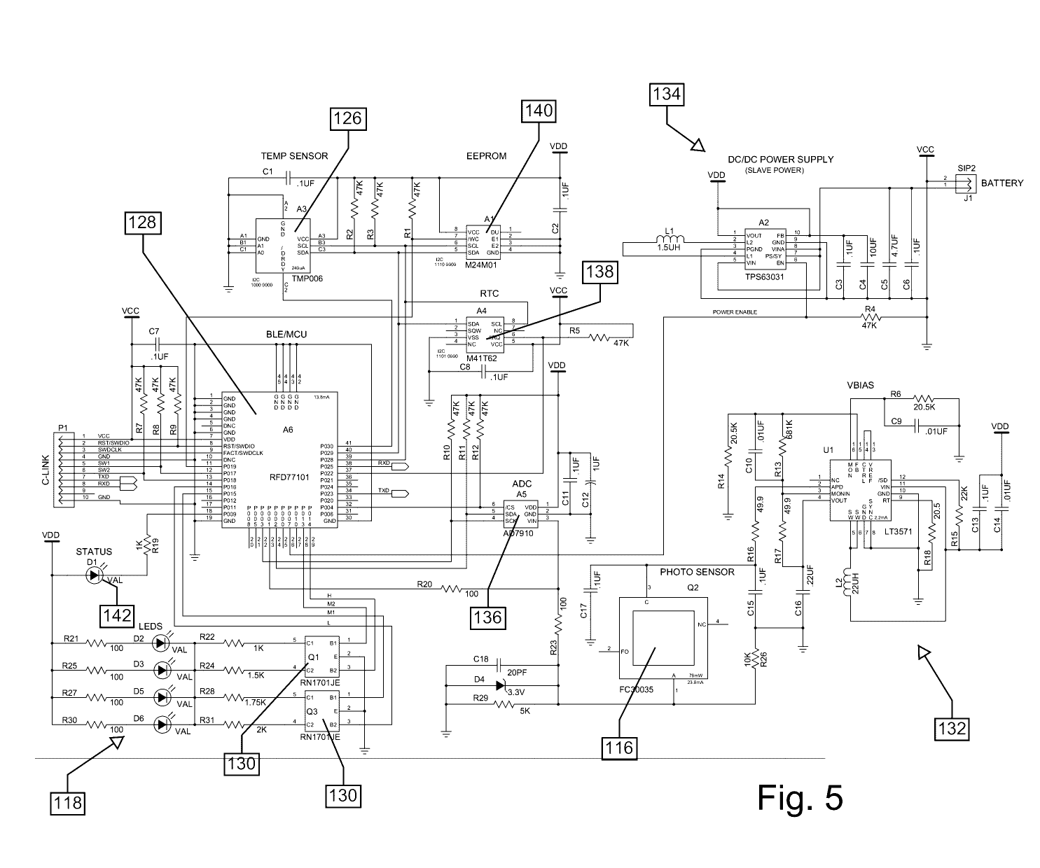

The overall arrangement and interconnection of the various electrical components is illustrated schematically in FIG. 5. In addition to the components identified above, the reader circuitry includes drivers 130, which control output of the distributed source of radiation 118. For example, the output may be regulated in steps, from 1 milliwatt to 1.5 milliwatt to 2 milliwatt to 2.5 milliwatt total output from the four LEDs comprising the distributed source of radiation 118. A bias-voltage control circuit 132 boosts power-supply voltage from the device power-supply circuit 134 to 27 volts for the photodetector 116 (which, as noted above, is suitably a photomultiplier) to use in generating photon-indicating signals. The power-supply circuit 134 is configured to operate with a source of DC voltage, e.g., a 3-volt coin-cell (or other) battery. Analog-to-digital converter 136 digitizes the output signal from the photodetector 116 for processing by the microprocessor 128, and real-time clock 138 provides date- and time-tracking functionality. EEPROM 140 provides storage for code, tracking of data, etc., and status indicator 142, which may be a simple LED, is configured to inform a user of the operational status of the device.

In some instances, a reader can interact with an implanted analyte sensor as shown schematically in FIG. 6. In general, it is known that light entering the skin will be scattered quickly (i.e., within a short distance from the surface) and be diffused within the skin. As a result, some known readers use a discrete source of excitation radiation to generate a small, highly localized “orb” of radiation within the skin, and that “orb” of radiation will only stimulate a small portion of the embedded sensor. Therefore, because only a percentage of the indicator molecules on the sensor will be stimulated by radiation at any given moment in time, it becomes necessary with such known readers to emit radiation for a longer period of time or to subject the sensor to more pulse-read cycles in order to obtain a sufficiently accurate read through large multi-sample-averaging of readings to obtain a meaningful measurement of analyst concentration. However, the indicator molecule population will gradually and eventually lose their ability to respond to stimulating radiation with continued exposure to radiation (photo-bleaching); when enough indicator molecules have lost their ability to respond that the sensor is no longer able to emit a photo-signal of sufficient signal-to-noise ratio that is strong enough to be discernible by the photodetector within the reader, the analyte sensor will have lost its useful life. Therefore, the need to emit stimulating radiation for longer periods of time—thus causing longer periods of light exposure for the indicator molecules—associated with some known readers is detrimental to the longevity of the analyte sensors they are configured to “read.”

Further still, with some known readers, only a portion of the analyte sensor is able to be read because the discrete source of radiation and the photodetector are located side-by-side on their supporting circuit board or within the overall optical assembly. As a result of this configuration, these two components cannot simultaneously be brought into their respective optimal positions with respect to an implanted analyte sensor, and only a portion of the sensor will be stimulated by the excitation radiation and only a portion of the emitted response radiation will be detected. Therefore, to overcome this situation (which leads to sub-optimal signal-to-noise ratios in the light being sensed/detected by the reader), the radiation source in known readers tends to be significantly overpowered, e.g., to put out as much as 200 milliwatts of illumination power. And that, in turn, can substantially reduce the useful life of the reader's batteries and/or charge intervals (in addition to reducing the longevity of the sensor, as noted above).

In contrast, a reader 100 has a distributed source of radiation 118, which at least partially surrounds the photodetector 116 and which is centered with respect to the photodetector 116. As a result, even in an embodiment such as the disclosed embodiment in which the distributed source of radiation 118 is formed from multiple individual sources of radiation (e.g., LEDs), the “orbs” of light 144 generated within the skin in the vicinity of each individual source will, due to scattering of light within the skin, merge into a photon “cloud” 146 that substantially envelopes the sensor 102, as illustrated in FIG. 6. And this, in turn, is advantageous for a number of reasons.

First, because the sensor 102 is substantially enveloped by the photon cloud 146, the indicator molecules over the entirety of the sensor 102 will participate in the analyte-concentration-indicating process. This significantly improves signal-to-noise ratio of the detected photo signal. Furthermore, because more indicator molecules participate in the process at any given moment in time, the sensor does not need to be stimulated for as long a period of time as is the case with known readers; as a result, the indicator molecules will not be photobleached nearly as quickly as is the case with respect to the known readers. Further still, because more indicator molecules participate in the process, and because the source of radiation is distributed relative to the sensor 102, the individual sources (i.e., the LEDs) do not need to be driven to put out light at as high a power level as is the case with respect to known readers.

Moreover, providing the source of radiation in distributed fashion facilitates centering the photodetector immediately over the sensor, where the photodetector can sense emitted response light as maximally as possible. Hence, in this way, too, the configuration of readers described herein can optimize sensitivity of the reader and accuracy of the readings taken with it.

The foregoing disclosure is intended to be by way of example only. Various modifications to and departures from the disclosed embodiment may occur to those having skill in the art without departing from the inventive concepts disclosed herein.

In one example, a reader in accordance can be configured to read tissue oxygen from a Wisniewski-type implant, which has been fabricated with an oxygen-sensitive indicator element where luminescent lifetime of the indicator is modulated by ambient tissue oxygen levels. In operation, synchronously pulsing the excitation array (i.e., the distributed source of radiation) results in a corresponding luminescent emission return pulse from the implant, with a signal-amplitude decay on the trailing edge of the pulse that is influenced quantitatively by the luminescent lifetime of the indicator. One method of signal processing suited for this application is commonly known as Time Domain, the definition of which is the time it takes for a step response such as a pulse to decrease in value to 1/e (˜36.8%) of its initial value (Io). Ratiometric signal-processing may also be used, where two channels are taken as a ratio with one being analyte-sensitive and the other not being analyte-sensitive for intensity-type (i.e., very-short-decay-time) indicator molecules.

Moreover, in some embodiments, the reader can be configured in various ways for different applications consistent with tissue-integrating implants or non-tissue-integrating photo-luminescent implants. For example, the device can be configured to operate at multiple excitation and/or emission wavelengths. More LEDs can be included in the surrounding array, and each wavelength can be selected individually or in combinations from the system electronics controller. LEDs may be single- or multi-color type constructs; SMT type; die; multi-die; or combinations. LEDs may be discrete within the array, or they may be configured through use of a transmissive waveguide into a ring type structure surrounding the detector for seamless distribution. Similarly, the detector can be a single-channel device or it may be divided into a multi-channel detector pair or quad (or smaller) with individual pass filters installed onto each detector segment. The photodetector can be a silicon photomultiplier chip (preferred) or a photodiode, a PIN diode, an avalanche photodiode, or any optical chip-based configuration. Optical filters may be high-pass, band-pass, thick- or thin-film, inorganic, organic constructs, or combinations thereof, and may be fabricated and installed by use of adhesives, sputtering, vacuum deposition, or combinations of these. It is also envisioned that the readers described herein can further be configured into pairs or more to create additional channels for even more analytes to be read simultaneously.

We claim:

1. An apparatus, comprising:
a reader substrate;
a photodetector configured to receive light emitted by a sensor implanted at an implantation depth within tissue, the photodetector coupled to a central portion of the reader substrate;
a plurality of emitters, each emitter from the plurality of emitters coupled to a peripheral portion of the reader substrate, each emitter configured to produce a far-field illumination pattern at the implantation depth, each emitter spaced apart from each other emitter such that, in the absence of optical scattering induced by the tissue, the collective far-field illumination pattern of the plurality of emitters has a central dark region at the implantation depth.

2. The apparatus of claim 1, wherein each emitter is spaced apart from each other emitter such that, in the absence of optical scattering induced by the tissue, the far-filed illumination pattern of that emitter does not overlap a far-field illumination pattern of any other emitter at the implantation depth.

3. The apparatus of claim 1, wherein the far-field illumination pattern produced by each emitter from the plurality of emitters has a diameter at the implantation depth that is less than a length of the sensor in the absence of optical scattering induced by the tissue.

4. The apparatus of claim 1, wherein the reader substrate includes an isolation portion disposed on perimeter portion of the reader substrate peripheral to the plurality of emitters, the isolation portion configured to reflect light from the plurality of emitters towards the photodetector.

5. The apparatus of claim 1, wherein the reader substrate further includes an isolation portion disposed between the photodetector and the plurality of emitters, the isolation portion configured to reduce light emitted from the plurality of emitters, and scattered by tissue before reaching the sensor, from reaching the photodetector.

6. The apparatus of claim 1, wherein the plurality of emitters are collectively configured to illuminate an entire length of the sensor when the sensor is implanted into tissue.

7. The apparatus of claim 1, wherein the plurality of emitters are collectively configured such that the far-field illumination patterns of at least two of the plurality of emitters overlap at the implantation depth within the tissue.

8. The apparatus of claim 1, wherein the implantation depth is between 2 mm and 5 mm.

9. The apparatus of claim 1, wherein the implantation depth is between 1 mm and 10 mm.

10. An apparatus, comprising:
a reader substrate;
a photodetector configured to receive light emitted by a sensor implanted within tissue, the photodetector coupled to a central portion of the reader substrate;
a plurality of emitters, each emitter from the plurality of emitters coupled to the reader substrate at a position radial to the central portion of the reader substrate; and
a optical isolation member coupled to the reader substrate at a position radial to the plurality of emitters, the optical isolation member configured to reflect light emitted from each of the plurality of emitters towards the central portion of the reader substrate.

11. The apparatus of claim 10, wherein each emitter from the plurality of emitters is configured to produce a far-field illumination pattern at the implantation depth, each emitter spaced apart from each other emitter such that the far-field illumination pattern of that emitter does not overlap a far-field illumination pattern of any other emitter at the implantation depth in the absence of optical scattering induced by the tissue.

12. The apparatus of claim 10, wherein the plurality of emitters are collectively configured to emit light having a far-field emission pattern at the implantation depth, the far-field emission pattern, in the absence of scattering, having a central dark region.

13. The apparatus of claim 10, wherein the plurality of emitters are collectively configured to emit light having a far-field emission pattern at the implantation depth, the far-field emission pattern, in the absence of scattering, having a central dark region, the plurality of emitters collectively configured to emit light having a far-filed emission pattern not having the central dark region in the presence of scattering induced by the tissue.

14. The apparatus of claim 10, wherein:
the plurality of emitters are collectively configured to emit light having a far-field emission pattern at the implantation depth, the far-field emission pattern, in the absence of scattering, having a central dark region; and
the photodetector is configured to receive the light emitted by the sensor in response the sensor being illuminated by the far-field emission pattern, the light received by the photodetector having an intensity associated with an entire length of the sensor being illuminated.

15. The apparatus of claim 10, wherein the optical isolation member is a first optical isolation member, the apparatus further comprising:
a second optical isolation member coupled to the reader substrate between the plurality of emitters and the photodetector.

16. A method, comprising:
emitting a first cone of light from a first emitter such that the first cone of light illuminates a first portion of a sensor disposed within tissue at an implantation depth, the first cone of light emitted from the first emitter such that, in the absence of scattering or reflection, the first cone of light has a diameter at the implantation depth that is less than a length of the sensor;
emitting a second cone of light from a second emitter such that the second cone of light illuminates a second portion of the sensor, the second cone of light emitted from the second emitter such that, in the absence of scattering or reflection, the second cone of light has a diameter at the implantation depth that is less than the length of the sensor, the second emitter spaced apart from the first emitter such that the first cone of light and the second cone of light, in the absence of scattering or reflection, do not overlap at the implantation depth;
receiving, at a photodetector, an emission signal from the sensor, the emission signal produced by the sensor in response the sensor being illuminated by the first cone of light and the second cone of light, the emission signal having an intensity associated with an entire length of the sensor being illuminated by the first cone of light and the second cone of light, collectively.

17. The method of claim 16, wherein the first cone of light and the second cone of light collectively illuminate the entire length of the sensor in the presence of at least one of scattering or reflection.

18. The method of claim 16, wherein the first cone of light and the second cone of light overlap at the implantation depth in the presence of scattering induced by the tissue.

19. The method of claim 16, wherein the first emitter and the second emitter are from a plurality of emitters, the method further comprising:
emitting, from the plurality of emitters, a plurality of cones of light, each emitter from the plurality of emitters spaced apart from each other emitter such that each cone of light from the plurality of cones of light does not overlap any other cone of light at the implantation depth in the absence of scattering or reflection.

20. The method of claim 16, further comprising:
reflecting, towards the photodetector, a portion of the first cone of light from a perimeter of a substrate coupled to the first emitter, the second emitter, and the photodetector.

21. The method of claim 16, wherein:
the photodetector is coupled to a central portion of a reader substrate;
the first emitter and the second emitter are coupled to the reader substrate on opposite sides of the photodetector; and
the first emitter and the second emitter are spaced apart at a distance that prevents the first cone of light and the second cone of light from overlapping at the implantation depth, in the absence of scattering or reflection.

22. The method of claim 16, wherein the first emitter and the second emitter are from a plurality of emitters, the method further comprising:
emitting, from the plurality of emitters, a plurality of cones of light, the plurality of cones of light having a collective far-field illumination pattern at the implantation depth having a central dark region at the implantation depth in the absence of scattering.
